(19) United States
(12) Patent Application Publication
(10) Pub. No.: US 2021/0291165 A1
(43) Pub. Date: Sep. 23, 2021

(54) RAPID DIAGNOSTIC TEST

(71) Applicant: Detect, Inc., Guilford, CT (US)

(72) Inventors: Jonathan M. Rothberg, Guilford, CT (US); Spencer Glantz, West Hartford, CT (US); Benjamin Rosenbluth, Hamden, CT (US); Todd Roswech, Ivoryton, CT (US); Matthew Dyer, Spring, TX (US); Jose Camara, Saratoga, CA (US); Eric Kauderer-Abrams, Redwood City, CA (US); Jonathan C. Schultz, Guilford, CT (US)

(73) Assignee: Detect, Inc., Guilford, CT (US)

(21) Appl. No.: 17/203,552

(22) Filed: Mar. 16, 2021

Related U.S. Application Data
(60) Provisional application No. 63/081,201, filed on Sep. 21, 2020.
(60) Provisional application No. 63/068,303, filed on Aug. 20, 2020.
(60) Provisional application No. 63/065,131, filed on Aug. 13, 2020.
(60) Provisional application No. 63/059,928, filed on Jul. 31, 2020.
(60) Provisional application No. 63/053,534, filed on Jul. 17, 2020.
(60) Provisional application No. 63/036,887, filed on Jun. 9, 2020.
(60) Provisional application No. 63/027,859, filed on May 20, 2020.
(60) Provisional application No. 63/027,874, filed on May 20, 2020.
(60) Provisional application No. 63/027,890, filed on May 20, 2020.
(60) Provisional application No. 63/027,864, filed on May 20, 2020.
(60) Provisional application No. 63/027,878, filed on May 20, 2020.
(60) Provisional application No. 63/027,886, filed on May 20, 2020.
(60) Provisional application No. 63/022,534, filed on May 10, 2020.
(60) Provisional application No. 63/022,533, filed on May 10, 2020.
(60) Provisional application No. 63/013,450, filed on Apr. 21, 2020.
(60) Provisional application No. 63/010,578, filed on Apr. 15, 2020.
(60) Provisional application No. 63/010,626, filed on Apr. 15, 2020.
(60) Provisional application No. 63/002,209, filed on Mar. 30, 2020.
(60) Provisional application No. 62/991,039, filed on Mar. 17, 2020.

(51) Int. Cl. B01L 3/00; B01L 7/00; G01N 33/543

(52) U.S. Cl. CPC B01L 3/50273; B01L 7/00; B01L 2200/027; B01L 2300/18; G01N 33/54386

(57) ABSTRACT

Provided herein, in some embodiments, are rapid diagnostic tests to detect one or more target nucleic acid sequences (e.g., a nucleic acid sequence of one or more pathogens). In some embodiments, the pathogens are viral, bacterial, fungal, parasitic, or protozoan pathogens, such as SARS-CoV-2 or an influenza virus. Further embodiments provide methods of detecting genetic abnormalities. Diagnostic tests comprising a sample-collecting component, one or more reagents (e.g., lysis reagents, nucleic acid amplification reagents), and a detection component (e.g., a component comprising a lateral flow assay strip and/or a colorimetric assay) are provided.

### CROSS-REFERENCE TO RELATED APPLICATIONS

The present application claims the benefit under 35 U.S.C. § 119(e) of U.S. Provisional Patent Application No. 62/991,039, filed Mar. 17, 2020 under Attorney Docket No. H0966.70014US00, titled “Viral Rapid Test,” U.S. Provisional Patent Application No. 63/002,209, filed Mar. 30, 2020 under Attorney Docket No. H0966.70014US01, titled “Viral Rapid Test,” U.S. Provisional Patent Application No. 63/010,578, filed Apr. 15, 2020 under Attorney Docket No. H0966.70014US02, titled “Viral Rapid Test,” U.S. Provisional Patent Application No. 63/010,626, filed Apr. 15, 2020 under Attorney Docket No. H0966.70014US03, titled “Viral Rapid Colorimetric Test,” U.S. Provisional Patent Application No. 63/013,450, filed Apr. 21, 2020 under Attorney Docket No. H0966.70014US04, titled “Method of Making and Using a Viral Test Kit,” U.S. Provisional Patent Application No. 63/022,534, filed May 10, 2020, under Attorney Docket No. H0966.70014US06, titled “Rapid Diagnostic Test,” U.S. Provisional Patent Application No. 63/022,533, filed May 10, 2020, under Attorney Docket No. H0966.70014US07, titled “Rapid Diagnostic Test,” U.S. Provisional Patent Application No. 63/036,887, filed Jun. 9, 2020, under Attorney Docket No. H0966.70014US08, titled “Rapid Diagnostic Test,” U.S. Provisional Patent Application No. 63/081,201, filed Sep. 21, 2020, under Attorney Docket No. H0966.70014US10, titled “Rapid Diagnostic Test,” U.S. Provisional Patent Application No. 63/065,131, filed Aug. 13, 2020, under Attorney Docket No. H0966.70014US11, titled “Apparatuses and Methods for Performing Rapid Diagnostic Tests,” U.S. Provisional Patent Application No. 63/059,928, filed Jul. 31, 2020 under Attorney Docket No. H0966.70014US12, titled “Rapid Diagnostic Test,” U.S. Provisional Patent Application No. 63/068,303, filed Aug. 20, 2020, under Attorney Docket No. H0966.70014US14, titled “Apparatuses and Methods for Performing Rapid Multiplexed Diagnostic Tests,” U.S. Provisional Patent Application No. 63/027,859, filed May 20, 2020, under Attorney Docket No. H0966.70014US15, titled “Rapid Self Administrable Test,” U.S. Provisional Patent Application No. 63/027,874, filed May 20, 2020, under Attorney Docket No. H0966.70014US16, titled “Rapid Self Administrable Test,” U.S. Provisional Patent Application No. 63/027,890, filed May 20, 2020, under Attorney Docket No. H0966.70014US17, titled “Rapid Self Administrable Test,” U.S. Provisional Patent Application No. 63/027,864, filed May 20, 2020, under Attorney Docket No. H0966.70014US18, titled “Rapid Self Administrable Test,” U.S. Provisional Patent Application No. 63/027,878, filed May 20, 2020, under Attorney Docket No. H0966.70014US19, titled “Rapid Self Administrable Test,” U.S. Provisional Patent Application No. 63/027,886, filed May 20, 2020, under Attorney Docket No. H0966.70014US20, titled “Rapid Self Administrable Test,” and U.S. Provisional Patent Application No. 63/053,534, filed Jul. 17, 2020, under Attorney Docket No. H0966.70014US22, titled “Computer Vision Algorithm For Diagnostic Testing,” each of which is hereby incorporated by reference in its entirety.

### FIELD

The present invention generally relates to diagnostic devices, systems, and methods for detecting the presence of a target nucleic acid sequence.

### BACKGROUND

The ability to rapidly diagnose diseases—particularly highly infectious diseases—is critical to preserving human health. As one example, the high level of contagiousness, the high mortality rate, and the lack of a treatment or vaccine for the coronavirus disease 2019 (COVID-19) have resulted in a pandemic that has already infected millions and killed hundreds of thousands of people. The existence of rapid, accurate COVID-19 diagnostic tests could allow infected individuals to be quickly identified and isolated, which could assist with containment of the disease. In the absence of such diagnostic tests, COVID-19 may continue to spread unchecked throughout communities.

### SUMMARY

Provided herein are a number of diagnostic tests useful for detecting target nucleic acid sequences. The tests, as described herein, are able to be performed in a point-of-care (POC) setting or home setting without specialized equipment. Therefore, in some aspects, the disclosure provides an integrated single-use device for performing a nucleic acid diagnostic test, the device comprising a lysis chamber for accepting a sample suspected of comprising a target nucleic acid sequence, wherein the lysis chamber comprises a lysis buffer, an amplification chamber operably connected to the lysis chamber through at least a first channel, a readout strip operably connected to the amplification chamber, and a pumping tool configured to be operated to transport at least some of the sample from the lysis chamber through the first channel and into the amplification chamber of the device.

In some aspects, the disclosure provides a system comprising the device and at least one computer readable medium comprising instructions that, when executed, cause a computing device to image the device and present results of the diagnostic test based on the image.

In some aspects, the disclosure provides a device comprising a cartridge comprising a lysis chamber for accepting a sample suspected of comprising a target nucleic acid sequence, wherein the lysis chamber comprises a lysis buffer, and an amplification chamber operably connected to the lysis chamber through at least a first channel of the cartridge, wherein the amplification chamber comprises lyophilized amplification reagents, wherein the first channel and the amplification chamber are enclosed within the cartridge, and a pumping tool configured to be operated to transport at least some of the sample from the lysis chamber through the first channel and into the amplification chamber of the device.

The foregoing apparatus and method embodiments may be implemented with any suitable combination of aspects, features, and acts described above or in further detail below. These and other aspects, embodiments, and features of the present teachings can be more fully understood from the following description in conjunction with the accompanying drawings.

### BRIEF DESCRIPTION OF THE DRAWINGS

Various aspects and embodiments will be described with reference to the following figures. It should be appreciated that the figures are not necessarily drawn to scale. In the drawings, each identical or nearly identical component that is illustrated in various figures is represented by a like numeral. For purposes of clarity, not every component may be labeled in every drawing.

FIG. 1 is a schematic view of a diagnostic device for performing a nucleic acid test, according to some embodiments;

FIG. 2 is a schematic view of a diagnostic device for performing a nucleic acid test comprising blister packs, according to some embodiments;

FIGS. 3A-3E depict a diagnostic cartridge comprising a first reservoir, a second reservoir, a third reservoir, a vent path, a detection region, and a pumping tool, according to some embodiments;

FIGS. 4A-4F depict a diagnostic cartridge comprising a first reservoir, a second reservoir, a third reservoir, a gas expansion reservoir, a detection region, and a pumping tool, according to some embodiments;

FIGS. 5A-5B depict a diagnostic cartridge comprising first, second, and third reservoirs comprising removable caps, according to some embodiments;

FIGS. 6A-6B depict a diagnostic cartridge comprising first and second reservoirs comprising removable caps, according to some embodiments;

FIGS. 7A-7D depict a diagnostic cartridge comprising a first reservoir, a second reservoir, and a wraparound pumping tool, according to some embodiments;

FIG. 8 depicts a diagnostic cartridge comprising first, second, and third reservoirs and a wraparound pumping tool, according to some embodiments; and

FIG. 9 depicts a diagnostic system comprising a sample-collecting component and a diagnostic cartridge, according to some embodiments.

### DETAILED DESCRIPTION

The present disclosure provides diagnostic devices, systems, and methods for rapidly and in a home environment detecting one or more target nucleic acid sequences (e.g., a nucleic acid sequence of a pathogen, such as SARS-CoV-2 or an influenza virus). A diagnostic system, as described herein, may be self-administrable and comprise a sample-collecting component (e.g., a swab) and a diagnostic device. The diagnostic device may comprise a cartridge that is pre-loaded with one or more reagents (e.g., lysis reagents, nucleic acid amplification reagents, CRISPR/Cas detection reagents) suitable for performing an assay. A user may operate the diagnostic device to move one or more of these solutions from one part of the cartridge to another to progress the assay, and eventually to produce a visual indication of a result of the assay.

As the COVID-19 pandemic has highlighted, there is a critical need for rapid, accurate systems and methods for diagnosing diseases—particularly infectious diseases. In the absence of diagnostic testing, asymptomatic infected individuals may unknowingly spread the disease to others, and symptomatic infected individuals may not receive appropriate treatment. With testing, however, infected individuals may take appropriate precautions (e.g., self-quarantine) to reduce the risk of infecting others and may receive targeted treatment as helpful.

While diagnostic tests for various diseases, including COVID-19, are known, such tests often require specialized knowledge of laboratory techniques and/or expensive laboratory equipment. For example, polymerase chain reaction (PCR) tests generally require skilled technicians and expensive, bulky thermocyclers. In addition, there is a need for diagnostic tests that are both rapid and highly accurate. Known diagnostic tests with high levels of accuracy often take hours, or even days, to return results, and more rapid tests generally have low levels of accuracy. Many rapid diagnostic tests detect antibodies, which generally can only reveal whether a person has previously had a disease, not whether the person has an active infection. In contrast, nucleic acid tests (i.e., tests that detect one or more target nucleic acid sequences) may indicate that a person has an active infection.

Diagnostic devices, systems, and methods described herein may be safely and easily operated or conducted by untrained individuals. Unlike prior art diagnostic tests, some embodiments described herein may not require knowledge of even basic laboratory techniques (e.g., pipetting). Similarly, some embodiments described herein may not require expensive laboratory equipment (e.g., thermocyclers). In some embodiments, reagents are contained within a reaction tube, a cartridge, and/or a blister pack, such that users are not exposed to any potentially harmful chemicals.

Diagnostic devices, systems, and methods described herein may also be highly sensitive and accurate. In some embodiments, the diagnostic devices, systems, and methods are configured to detect one or more target nucleic acid sequences using nucleic acid amplification (e.g., an isothermal nucleic acid amplification method). Through nucleic acid amplification, the diagnostic devices, systems, and methods are able to accurately detect the presence of extremely small amounts of a target nucleic acid. In certain cases, for example, the diagnostic devices, systems, and methods can detect 1 pM or less, or 10 aM or less.

As a result, the diagnostic devices, systems, and methods described herein may be useful in a wide variety of contexts. For example, in some cases, the diagnostic devices and systems may be available over the counter for use by consumers. In such cases, untrained consumers may be able to self administer the diagnostic test (or administer the test to friends and family members) in their own homes (or any other location of their choosing). In some cases, the diagnostic devices, systems, or methods may be operated or performed by employees or volunteers of an organization (e.g., a school, a medical office, a business). For example, a school (e.g., an elementary school, a high school, a university) may test its students, teachers, and/or administrators, a medical office (e.g., a doctor's office, a dentist's office) may test its patients, or a business may test its employees for a particular disease. In each case, the diagnostic devices, systems, or methods may be operated or performed by the test subjects (e.g., students, teachers, patients, employees) or by designated individuals (e.g., a school nurse, a teacher, a school administrator, a receptionist).

In some embodiments, diagnostic devices described herein are relatively small. In certain cases, for example, a cartridge is approximately the size of a pen or a marker. Thus, unlike diagnostic tests that require bulky equipment, diagnostic devices and systems described herein may be easily transported and/or easily stored in homes and businesses. In some embodiments, the diagnostic devices and systems are relatively inexpensive. Since no expensive laboratory equipment (e.g., a thermocycler) is required, diagnostic devices, systems, and methods described herein may be more cost effective than known diagnostic tests.

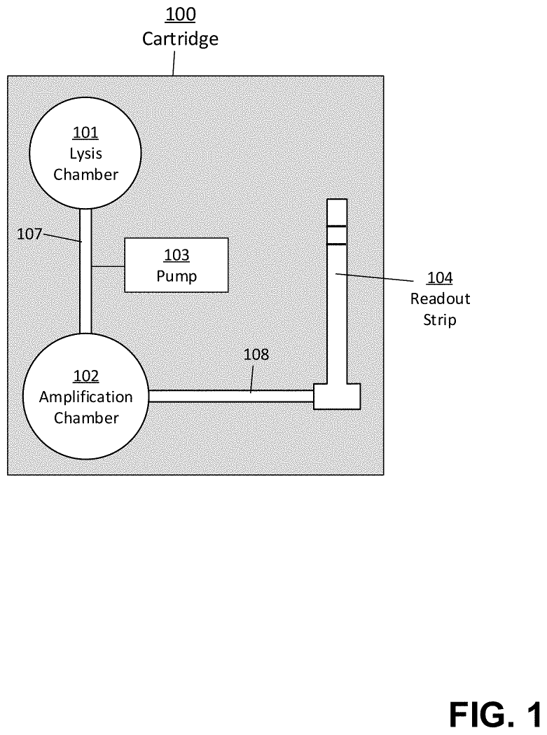

FIG. 1 is a schematic view of a diagnostic device for performing a nucleic acid test, according to some embodiments. In the example of FIG. 1, the diagnostic device is, or comprises, a cartridge 100 that includes a lysis chamber 101 coupled to an amplification chamber 102 via a channel 107. A pump 103 is also coupled to the channel 107. The amplification chamber 102 is coupled to a readout strip 104 via a channel 108. In some embodiments, the cartridge 100 may include, or may be coupled to, one or more heaters that may be operated to heat the lysis chamber 101 and/or amplification chamber 102.

During use of the cartridge 100, a user may obtain a sample-collecting component (e.g., a swab) containing a sample suspected to contain a target nucleic acid sequence and insert the component into the lysis chamber 101. Subsequent to lysing of the sample, which is described further below, the pump 103 may be operated, whether manually by the user and/or automatically by the device, to move liquid from the lysis chamber to the amplification chamber 102. As a result, the lysed sample may be transferred to react with the contents of the amplification chamber 102. Subsequent to amplification of the lysed sample, which is described further below, at least some contents of the amplification chamber 102 may be moved to the readout strip 104. The amplified and lysed sample may be transferred to the readout strip by the pump 103, via an additional pump (not shown in FIG. 1), and/or through capillary action that draws liquid from the amplification chamber onto the readout strip. The readout strip may contain reagents that detect whether the target nucleic acid sequence is present, and produce a visual indication thereof that may be viewed by a user. For instance, part of the readout strip may be visible through a window of the cartridge 100.

In terms of collecting the sample for use with cartridge 100 as part of a diagnostic method, in some embodiments the diagnostic method comprises collecting a sample from a subject (e.g., a human subject, an animal subject). Illustrative sample types may include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, the sample comprises a nasal secretion. In certain instances, for example, the sample is an anterior nares specimen. An anterior nares specimen may be collected from a subject by inserting a swab element of a sample-collecting component into one or both nostrils of the subject for a period of time. In some embodiments, the sample comprises a cell scraping. In some embodiments, the cell scraping is collected from the mouth or interior cheek. The cell scraping may be collected using a brush or scraping device formulated for this purpose. The sample may be self-collected by the subject or may be collected by another individual (e.g., a family member, a friend, a coworker, a health care professional) using a sample-collecting component described herein.

In some embodiments, the sample comprises an oral secretion (e.g., saliva). In certain cases, the volume of saliva in the sample is at least 1 mL, at least 1.5 mL, at least 2 mL, at least 2.5 mL, at least 3 mL, at least 3.5 mL, or at least 4 mL. In some embodiments, the volume of saliva in the sample is in a range from 1 mL to 2 mL, 1 mL to 3 mL, 1 mL to 4 mL, or 2 mL to 4 mL. Saliva has been found to have a mean concentration of SARS-Cov-2 RNA of 5 fM (Kai-Wang To et al., 2020)—an amount that is detectable by any one of the methods described herein.

The sample, in some embodiments, may be collected from a subject who is suspected of having the disease(s) the test screens for, such as a coronavirus (e.g., COVID-19) and/or influenza (e.g., influenza type A or influenza type B). Other indications, as described herein, are also envisioned. In some embodiments, the subject is a human. Subjects may be asymptomatic, or may present with one or more symptoms of the disease(s). Symptoms of coronaviruses (e.g., COVID-19) include, but are not limited to, fever, cough (e.g., dry cough), generalized fatigue, sore throat, headache, loss of taste or smell, runny nose, nasal congestion, muscle aches, and difficulty breathing (shortness of breath). Symptoms of influenza include, but are not limited to, fever, chills, muscle aches, cough, congestion, runny nose, headaches, and generalized fatigue. In some embodiments, the subject is asymptomatic, but has had contact within the past 14 days with a person that has tested positive for the virus.

As discussed above, in the example of FIG. 1 once the sample is collected it may be inserted into the lysis chamber 101. In some cases, the lysis chamber may include a seal (e.g., a foil seal) that may be broken by the sample collecting component when it is inserted into the lysis chamber.

In some embodiments, lysis is performed within lysis chamber 101 by chemical lysis (e.g., exposing a sample to one or more lysis reagents) and/or by thermal lysis (e.g., heating a sample in the case where cartridge 100 includes, or is coupled to, a heater). Chemical lysis may be performed by one or more lysis reagents supplied within the lysis chamber 101. As used herein, a lysis reagent generally refers to a reagent that promotes cell lysis either alone or in combination with one or more reagents and/or conditions (e.g., heating). In some cases, the one or more lysis reagents comprise one or more enzymes. Non-limiting examples of suitable enzymes include lysozyme, lysostaphin, zymolase, cellulose, protease, and glycanase. In some embodiments, the one or more lysis reagents comprise one or more detergents. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, the one or more lysis reagents comprise an RNase inhibitor (e.g., a murine RNase inhibitor).

In some embodiments, cell lysis is accomplished within lysis chamber 101 by applying heat to a sample (thermal lysis). In certain instances, thermal lysis is performed by applying a lysis heating protocol comprising heating the sample at one or more temperatures for one or more time periods using any heater described herein. In some embodiments, a lysis heating protocol comprises heating the sample at a given temperature for a desired time period. This may be repeated so that the heating protocol heats the sample at a first temperature for a first time period, then at a second temperature for a second time period, etc. Any of these temperatures may for instance be at least 37° C., at least 50° C., at least 60° C., at least 65° C., at least 70° C., at least 80° C., or at least 90° C. In certain instances, any of these time periods may be at least 1 minute, at least 2 minutes, at least 5 minutes, at least 10 minutes, at least 15 minutes, at least 20 minutes, or at least 30 minutes.

As discussed above, in the example of FIG. 1 once the sample is lysed, the lysed sample may be moved into the amplification chamber 102 through operation of the pump 103. As a result, one or more target nucleic acids (e.g., a nucleic acid of a target pathogen) may be amplified in the chamber 102.

DNA may be amplified in amplification chamber 102 according to any suitable nucleic acid amplification method. In some embodiments, the nucleic acid amplification method is an isothermal amplification method. Isothermal amplification methods include, but are not limited to, loop-mediated isothermal amplification (LAMP), recombinase polymerase amplification (RPA), nicking enzyme amplification reaction (NEAR), nucleic acid sequence-based amplification (NASBA), strand displacement amplification (SDA), helicase-dependent amplification (HDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In one embodiment, the nucleic acid amplification method is loop-mediated isothermal amplification (LAMP). In another embodiment, the nucleic acid amplification method is recombinase polymerase amplification (RPA). In another embodiment, the nucleic acid amplification method is nicking enzyme amplification reaction. The amplification chamber 102 may comprise one or more reagents suitable for performing any of the above amplification methods. For instance, where the amplification method includes LAMP, the amplification chamber 102 may comprise multiple primers selected for amplification of a particular nucleic acid sequence (e.g., primers for amplification of a SARS-CoV-2 nucleic acid sequence may be selected from regions of the virus's nucleocapsid (N) gene, envelope (E) gene, membrane (M) gene, and/or spike (S) gene).

In some embodiments, the device of FIG. 1 may be configured to perform an isothermal amplification method in which the device operates a heater to apply heat to a sample (e.g., via a heater within, or coupled to, cartridge 100). In certain instances, the device may be configured to apply an amplification heating protocol comprising heating the sample at one or more temperatures for one or more time periods using any heater described herein. In some embodiments, an amplification heating protocol comprises heating the sample at a given temperature for a desired time period. This may be repeated so that the heating protocol heats the sample at a first temperature for a first time period, then at a second temperature for a second time period, etc. Any of these temperatures may for instance be at least 30° C., at least 32° C., at least 37° C., at least 50° C., at least 60° C., at least 65° C., at least 70° C., at least 80° C., or at least 90° C. In certain instances, any of these time periods may be at least 1 minute, at least 2 minutes, at least 3 minutes, at least 4 minutes, at least 5 minutes, at least 10 minutes, at least 15 minutes, at least 20 minutes, or at least 30 minutes.

As discussed above, in the example of FIG. 1 once the sample is lysed and amplified, the amplified nucleic acids from the sample (e.g., amplicons) may be moved onto the readout strip 104 where one or more target nucleic acid sequences may be detected. In some embodiments, the readout strip 104 may be, or may comprise, a lateral flow assay strip. In some embodiments, the readout strip 104 may be configured to perform a colorimetric assay.

In some embodiments, the readout strip 104 may comprise a sub-region comprising a plurality of labeled particles, such as gold nanoparticles (e.g., colloidal gold nanoparticles) that produce labeled amplicons by binding labeled particles with the amplified nucleic acids from the sample. Non-limiting examples of suitable labels include biotin, streptavidin, fluorescein isothiocyanate (FITC), fluorescein amidite (FAM), fluorescein, and digoxigenin (DIG).

In some embodiments, the readout strip 104 may comprise one or more test lines. A test line may include a capture reagent (e.g., an immobilized antibody) configured to detect a target nucleic acid. For instance, a particle-amplicon conjugate within the readout strip may be captured by one or more capture reagents (e.g., immobilized antibodies), and an opaque marking may appear on the readout strip. The marking may have any suitable shape or pattern (e.g., one or more straight lines, curved lines, dots, squares, check marks, x marks). The readout strip may comprise multiple test lines that are each configured to detect a different target nucleic acid. Some of the test lines may be control lines that are configured to detect a human (or animal) nucleic acid control. For example, a human (or animal) nucleic acid control line may be configured to detect a nucleic acid (e.g., RNase P) that is generally present in all humans (or animals). In some cases, the human (or animal) nucleic acid control line becoming detectable indicates that a human (or animal) sample was successfully collected, nucleic acids from the sample were amplified, and the amplicons were transported through the lateral flow assay strip.

In some embodiments, the readout strip 104 comprises a sub-region to absorb fluid flowing through the lateral flow assay strip (e.g., a wicking area).

As an illustrative example of the above-described detection process that may be performed on the readout strip 104, a fluidic sample comprising an amplicon labeled with biotin and FITC may be introduced into the readout strip. As the labeled amplicon is transported through the lateral flow assay strip (e.g., through a particle conjugate pad of the lateral flow assay strip), a gold nanoparticle labeled with streptavidin may bind to the biotin label of the amplicon. The readout strip 104 may comprise a first test line comprising an anti-FITC antibody. In some embodiments, the gold nanoparticle-amplicon conjugate may be captured by the anti-FITC antibody, and an opaque band may develop as additional gold nanoparticle-amplicon conjugates are captured by the anti-FITC antibodies of the first test line. The readout strip 104 may further comprise a first lateral flow control line comprising biotin. Excess gold nanoparticles labeled with streptavidin (i.e., gold nanoparticles that were not conjugated to an amplicon) transported through the readout strip may bind to the biotin of the first lateral flow control line, demonstrating that liquid was successfully transported to the first lateral flow control line.

As another example of the above-described detection process that may be performed on the readout strip 104, one or more target nucleic acid sequences may be detected using a colorimetric assay. In some embodiments, for example, a fluidic sample may be exposed to a reagent that undergoes a color change when bound to a target nucleic acid (e.g., viral DNA or RNA), such as with an enzyme-linked immunoassay. In some embodiments, the assay further comprises a stop reagent, such as sulfonic acid. That is, when the fluidic sample is mixed with the reagents, the solution turns a specific color (e.g., red) if the target nucleic acid is present, and the sample is positive. If the solution turns a different color (e.g., green), the target nucleic acid is not present, and the sample is negative. In some embodiments, the colorimetric assay may be a colorimetric LAMP assay; that is, the LAMP reagents may react in the presence or absence of a target nucleic acid sequence (e.g., from SARS-CoV-2) to turn one of two colors.

In some embodiments, the readout strip 104 comprises one or more reagents for CRISPR/Cas detection. CRISPR generally refers to Clustered Regularly Interspaced Short Palindromic Repeats, and Cas generally refers to a particular family of proteins. In some embodiments, the CRISPR/Cas detection platform can be combined with an isothermal amplification method to create a single step reaction (Joung et al., “Point-of-care testing for COVID-19 using SHERLOCK diagnostics,” 2020). For example, the amplification and CRISPR detection may be performed using reagents having compatible chemistries (e.g., reagents that do not interact detrimentally with one another and are sufficiently active to perform amplification and detection). In some embodiments, CRISPR/Cas detection is combined with LAMP in the amplification chamber as described above. Examples of CRISPR/Cas detection platforms include SHERLOCK® and DETECTR® (see, e.g., Kellner et al., Nature Protocols, 2019, 14: 2986-3012; Broughton et al., Nature Biotechnology, 2020; Joung et al., 2020).

In some embodiments, the readout strip 104 is configured to perform one or more CRISPR/Cas techniques to detect a target nucleic acid sequence (e.g., from a pathogen). In particular, readout strip 104 may comprise a guide RNA (gRNA) designed to recognize a specific target sequence (e.g., a SARS-CoV-2-specific sequence) to detect target nucleic acid sequences present in a sample. If the sample comprises the target nucleic acid sequence, the gRNA will bind the target nucleic acid sequence and activate a programmable nuclease (e.g., a Cas protein), which may then cleave a reporter molecule and release a detectable signal (e.g., a reporter molecule tagged with specific antibodies for the lateral flow test, a fluorophore, a dye, a polypeptide, or a substrate for a specific colorimetric dye). In some embodiments, the detectable moiety binds to a capture reagent (e.g., an antibody) on the readout strip.

In some embodiments, the one or more reagents for CRISPR/Cas detection comprise one or more guide nucleic acids. As noted above, a guide nucleic acid may comprise a segment with reverse complementarity to a segment of the target nucleic acid sequence. In some embodiments, the guide nucleic acid is selected from a group of guide nucleic acids that have been screened against the nucleic acid of a strain of an infection or genomic locus of interest. In certain instances, for example, the guide nucleic acid may be selected from a group of guide nucleic acids that have been screened against the nucleic acid of a strain of SARS-CoV-2. In some embodiments, guide nucleic acids that are screened against the nucleic acid of a target sequence of interest can be pooled. Without wishing to be bound by a particular theory, it is thought that pooled guide nucleic acids directed against a single target nucleic acid can ensure broad coverage of the target nucleic acid within a single reaction. The pooled guide nucleic acids, in some embodiments, are directed to different regions of the target nucleic acid and may be sequential or non-sequential.

In some embodiments, a guide nucleic acid comprises a crRNA and/or tracrRNA. The guide nucleic acid may not be naturally occurring and may be made by artificial combination of otherwise separate segments of sequence. For example, in some embodiments, an artificial guide nucleic acid may be synthesized by chemical synthesis, genetic engineering techniques, and/or artificial manipulation of isolated segments of nucleic acids.

In some embodiments, the one or more reagents for CRISPR/Cas detection comprise one or more programmable nucleases. In some embodiments, a programmable nuclease is capable of sequence-independent cleavage after the gRNA binds to its specific target sequence. In some instances, the programmable nuclease is a Cas protein. Non-limiting examples of suitable Cas proteins include Cas9, Cas12a, Cas12b, Cas13, and Cas14. In general, Cas9 and Cas12 nucleases are DNA-specific, Cas13 is RNA-specific, and Cas14 targets single-stranded DNA.

In some embodiments, the one or more reagents for CRISPR/Cas detection comprise a plurality of guide nucleic acids and a plurality of programmable nucleases. In some embodiments, each guide nucleic acid of the plurality of guide nucleic acids targets a different nucleic acid and is associated with a different programmable nuclease. As an illustrative example, if a diagnostic device is configured to detect two different target nucleic acids, the one or more CRISPR/Cas reagents may comprise at least two different guide nucleic acids and at least two different programmable nucleases. If two target nucleic acids are present in a sample, then two different programmable nucleases will be activated, which will result in the release of two unique detectable moieties. Thus, in this manner, the CRISPR/Cas detection system may be used to detect more than one target nucleic acid. In some embodiments, the CRISPR/Cas detection system may be used to detect at least 2, at least 3, at least 4, at least 5, at least 6, at least 7, at least 8, at least 9, or at least 10 target nucleic acids.

In some embodiments, the readout strip 104 comprises a plurality of fibers (e.g., woven or non-woven fabrics). In some embodiments, the one or more fluid-transporting layers comprise a plurality of pores. In some embodiments, pores and/or interstices between fibers may advantageously facilitate fluid transport (e.g., via capillary action).

In some embodiments, the diagnostic device of FIG. 1 may comprise one or more blister packs. In some embodiments, a blister pack comprises one or more chambers. In some cases, each chamber may comprise one or more reagents (e.g., lysis reagents, nucleic acid amplification reagents) and/or one or more buffers (e.g., dilution buffer). A chamber may be separated from an adjacent chamber by a breakable seal (e.g., a frangible seal) or a valve (e.g., a rotary valve).

Diagnostic devices and systems described herein may comprise any number of blister packs, arranged in such a way so as to process a sample as described herein. In some embodiments, the blister packs comprise one or more seals (e.g., differential seals, frangible seals) that allow reagents to be delivered in a controlled manner (e.g., using differential seal technology). In some embodiments, the blister packs comprise one or more chambers, where each chamber comprises one or more reagents. In some embodiments, one or more chambers store one or more reagents in solid form (e.g., lyophilized, dried, crystallized, air jetted), and one or more chambers store one or more reagents and/or buffers in liquid form. In some cases, a chamber comprising one or more reagents in solid form may be separated from a chamber comprising one or more reagents and/or buffers in liquid form by a seal (e.g., a frangible seal). In some cases, breaking the frangible seal may result in the solid reagents being suspended in the one or more liquid reagents and/or buffers. In some cases, the suspended solid reagents may be added to a sample.

In some embodiments, the delivery of each reagent in a blister pack is fully automated. For example, the user may insert a sample in a sample collection region of the blister pack and then activate the blister pack. Upon activation, all of the reagents may be added to the sample in the correct amount and at the appropriate time, such that the sample is processed as described herein. In some embodiments, the blister pack further comprises a detection component (e.g., a lateral flow assay strip, a colorimetric assay). The detection component may alert the user as to whether the sample was positive or negative for the target nucleic acid sequence.

While in the example of FIG. 1 the various components have been shown as part of a single cartridge, a diagnostic device may not necessarily be configured as such, as particular components may be provided within the device but separate from the cartridge in some embodiments. For instance, the pump 103 may be separated from the cartridge 110 but coupled to the cartridge and the channel 107 on the cartridge so that it can be operated to move fluid as described above. Similarly, the readout strip 104 may be separate from the cartridge but fluidically coupled to the cartridge and channel 108 so that fluid may be moved off the cartridge and onto the strip.

The body of the cartridge 100 may be formed from any suitable material. Non-limiting examples of suitable materials include polymers (e.g., thermoplastic polymers) and metals. In some cases, the body of the cartridge is formed by injection molding, an additive manufacturing process (e.g., 3D printing), and/or a subtractive manufacturing process (e.g., laser cutting). In some embodiments, the device may include a seal plate coupled to the cartridge, which may be formed from suitable materials such as glass epoxy (e.g., FR4/G10), polymers (e.g., thermoplastic polymers), and metals. In some cases, a seal plate may be attached to the body of the cartridge by one or more fasteners (e.g., screws, nails, clamps, and/or bolts), one or more adhesives, and/or one or more interconnecting parts.

In some embodiments, the cartridge 100 may include additional fluid chambers that contain additional fluids such one or more buffers, reagent stability additives, etc. Such chambers may be coupled to the depicted channels and chambers and their contents combined with the lysed sample, or lysed and amplified sample, etc. using the pump 103 and/or some other device.

In embodiments in which the cartridge 100 includes, or is coupled to, one or more heaters, each heater may be configured to heat one or more components of the cartridge (e.g., fluidic contents of a reaction tube or a reservoir) to a plurality of temperatures for a plurality of time periods. Each heater may be pre-programmed with one or more protocols. For example, a heater may be pre-programmed with a lysis heating protocol and/or an amplification heating protocol. A lysis heating protocol generally refers to a set of one or more temperatures and one or more time periods that facilitate lysis of the sample. An amplification heating protocol generally refers to a set of one or more temperatures and one or more time periods that facilitate nucleic acid amplification. In some embodiments, the heater comprises an auto-start mechanism that corresponds to the temperature profile needed for lysis and/or amplification. That is, a user may initialize the device and the heater may, in response, automatically run a lysis and/or amplification heating protocol. In some embodiments, the heater is controlled by a mobile application.

In some embodiments, the diagnostic device comprising cartridge 100 may be part of a diagnostic system that comprises instructions for using the diagnostic device. The instructions may for instance be provided as part of a software-based application, such as an application on a portable computing device such as a smartphone or tablet, and which guides a user through steps to use the diagnostic device. In some embodiments, the instructions instruct a user when to add certain reagents and how to do so. For example, in certain instances, the instructions may instruct a user how to operate the pump to move reagents from one part of the cartridge to another (e.g., by depressing a button, twisting a portion of the reaction tube cap, etc.). In some embodiments, the instructions instruct a user on beginning and/or ending heating protocols. In some cases, a user may receive an alert (e.g., on a mobile application) when a heating protocol (e.g., a lysis heating protocol, an amplification heating protocol) is complete. In some embodiments, the application may validate that the diagnostic test was performed correctly.

In some embodiments, a software-based application may be connected (e.g., via a wired or wireless connection) to one or more components of a diagnostic system. In some embodiments, for example, a heater may be controlled by a software-based application. In some cases, a user may select an appropriate heating protocol through the software-based application. In some cases, an appropriate heating protocol may be selected remotely (e.g., not by the immediate user). In some cases, the software-based application may store information (e.g., regarding temperatures used during the processing steps) from the heater.

In some embodiments, a diagnostic systems comprises or is associated with software to read and/or analyze test results. In some embodiments, a device (e.g., a camera, a smartphone) is used to generate an image of a test result (e.g., one or more lines detectable on a lateral flow assay strip). In certain cases, a machine vision software application is employed to evaluate the image and provide a positive or negative test result. That result may be communicated directly to a user or to a medical professional. In some cases, the test result may be further communicated to a remote database server. In some embodiments, the remote database server stores test results as well as user information. For example, the remote database server may store at least one of name, social security number, date of birth, address, phone number, email address, medical history, and medications.

In some embodiments, the remote database server may track and monitor locations of users (e.g., using smartphones or remote devices with tracking capabilities). In some cases, the remote database server can be used to notify individuals who come into contact with or within a certain distance of any user who has tested positive for a particular illness (e.g., COVID-19). In some cases, a user's test results, information, and/or location may be communicated to state and/or federal health agencies.

In some embodiments, a user may use an electronic device (e.g., a smartphone, a tablet, a camera) to acquire an image of some or all of readout strip 104. In some embodiments, software running on the electronic device may analyze the image (e.g., by comparing any lines or other markings that appear on the lateral flow assay strip with known patterns of markings).

After uploading the image, the computing device may perform a computer vision algorithm to electronically call the bands. If the band-pattern result determined by the algorithm differs from the band pattern result entered by the user, the user is asked to double-check that they entered the correct band-pattern, and the user is given the opportunity to redo to the “Record Results” page. Alternatively, in some embodiments, the interpretation is performed solely by the computer-vision algorithm. Based on the result that the user entered, the user is shown the corresponding “Test Complete” screen in the mobile application, which tells the user if the test result is positive, negative, or invalid. In addition to providing the test result, careful language is used to ensure that the user can properly interpret the meaning of the result.

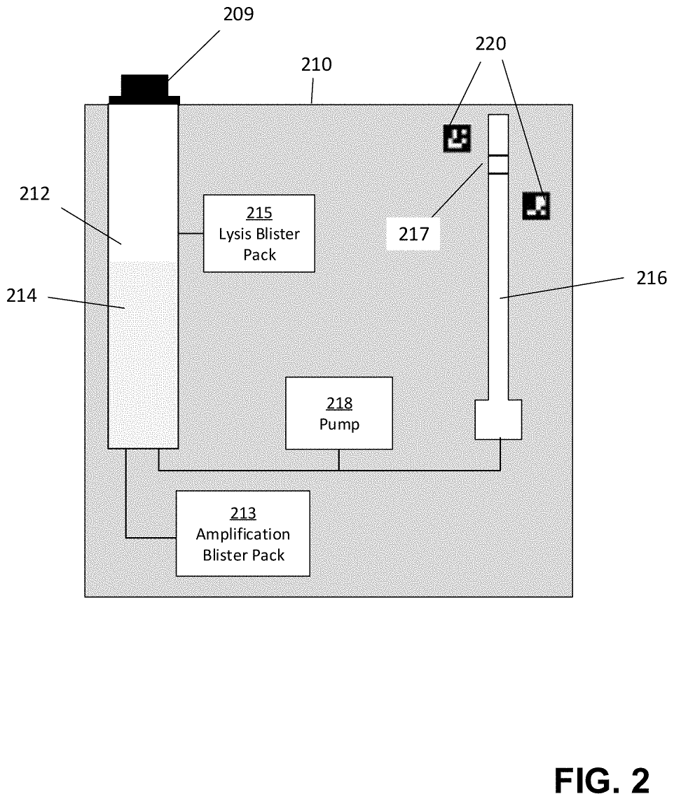

FIG. 2 is a schematic view of a diagnostic device for performing a nucleic acid test that includes blister packs, according to some embodiments. To provide another example of a suitable diagnostic device similar to that of FIG. 1, FIG. 2 depicts a diagnostic device that is, or includes, cartridge 210. The cartridge 210 includes a tube 212 containing a reaction buffer 214, which is coupled to blister packs 213 and 215. In some embodiments, the cartridge 210 may also comprise, or may be coupled to, a heater in thermal communication with tube 212.

In operation of the cartridge 210, a sample may be added through sample port 209 so that the sample is supplied into the reaction buffer 214. For example, a sample-collecting component (e.g., a swab) containing a sample may be inserted into the tube 212. Subsequently, a lysis blister pack 215 comprising one or more lysis and/or decontamination reagents (e.g., UDG) may be released from the blister pack 215 into tube 212. Release of the contents of the blister pack 215 may be performed in a manual or automated manner via any of the techniques discussed above (e.g., by a user breaking a frangible seal, etc.). In some embodiments, the cartridge 210 may comprise, or may be coupled to, a heater configured to heat tube 212 (a heater is not shown in FIG. 2).

Subsequent to release of the blister pack 215, one or more amplification reagents may be released from amplification blister pack 213 into tube 212. Release of the contents of the blister pack 213 may be performed in a manual or automated manner via any of the techniques discussed above (e.g., by a user breaking a frangible seal, etc.). The contents of the tube 212 may then be flowed onto the lateral flow assay strip 216 through operation of pump 218 to draw liquid from the tube onto the strip 216. As discussed above, the strip may react with amplified DNA present in the lysed and amplified sample and produce a visual indication of whether a target DNA sequence was detected. In the example of FIG. 2, test lines 217 on the strip 216 may be visible, or not, depending on whether such targets were detected (with each test line's appearance or absence indicating whether or not a respective target was detected). Cartridge 210 also includes fiducial markers 220 that may allow an imaging device to align to the test lines 217 and detect their presence of absence. The markers 220 may for instance comprise QR barcodes that may encode device information and may be used by a software-based application (e.g., to pair the user to the test result).

Additional practical implementations of the diagnostic devices of FIG. 1 and/or FIG. 2 are further described below. It will be appreciated that in each case the various lysis, amplification and detection techniques discussed above in relation to FIG. 1 may be embodied by these devices and applied during operation of the devices. In particular, any of these devices may include a readout strip that comprises one or more CRISPR/Cas reagents for detection of a target DNA sequence using CRISPR detection techniques.

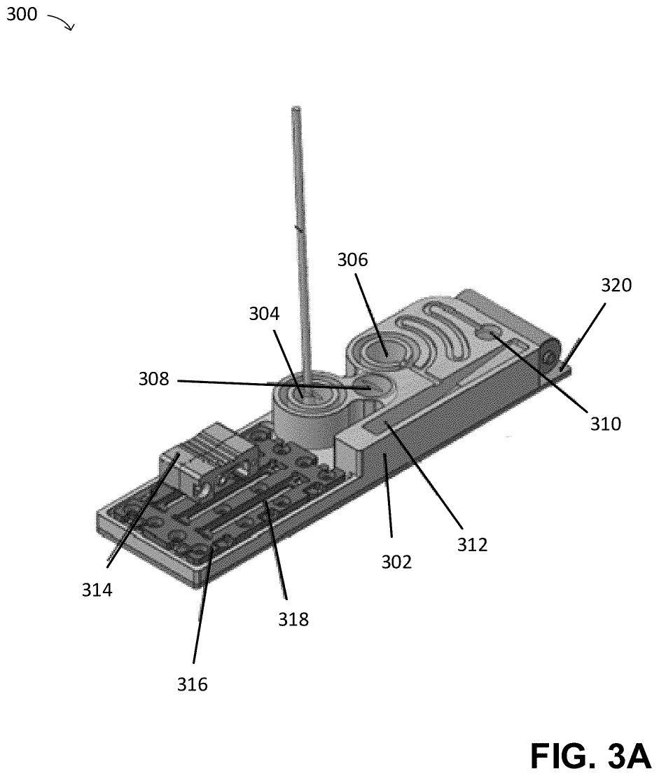

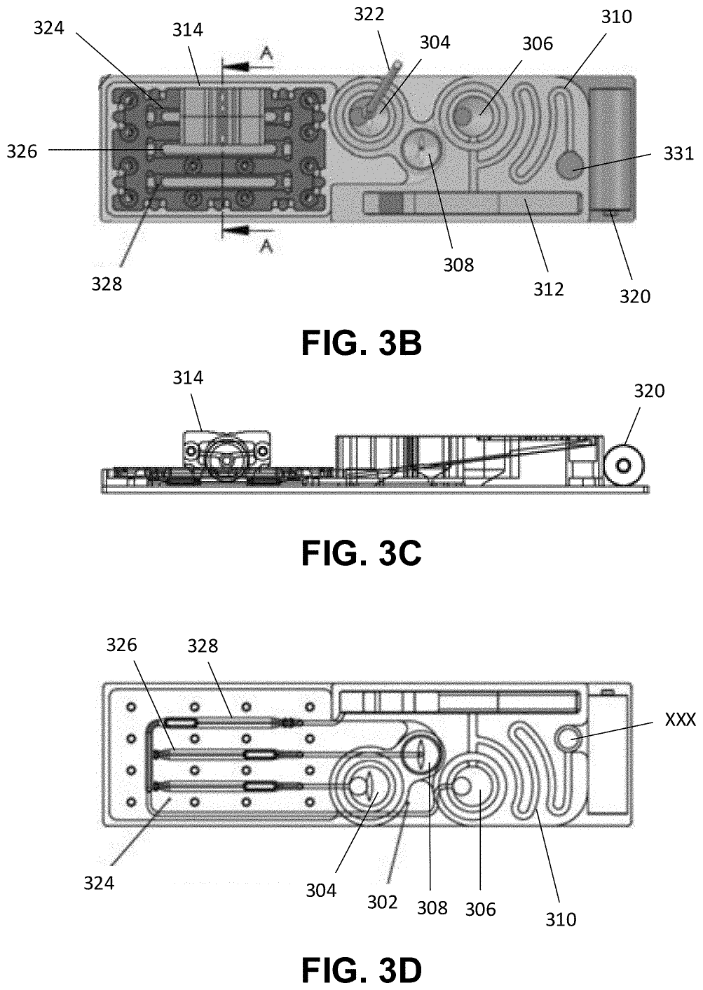

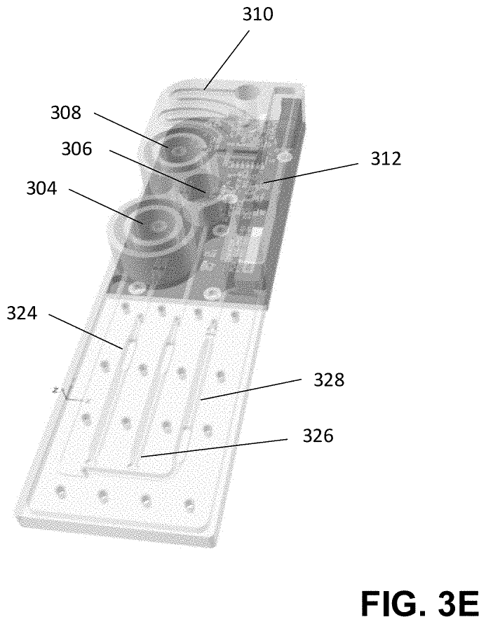

One such illustrative diagnostic device is shown in FIGS. 3A-3E. FIG. 3A shows a top perspective view of the device, FIG. 3B a top view, FIG. 3C a side view, FIG. 3D a back view, and FIG. 3E an additional view of the cartridge body and integrated heater. In the example of FIGS. 3A-3E, cartridge 300 comprises cartridge body 302, which may be formed from any suitable material (e.g., a moldable thermoplastic material). As shown in FIG. 3A, cartridge body 302 comprises first reagent reservoir 304, second reagent reservoir 306, third reagent reservoir 308, vent path 310, and detection region 312. In some cases, first reagent reservoir 304, second reagent reservoir 306, and third reagent reservoir 308 are each fluidically connected (e.g., directly fluidically connected) to one or more fluidic channels (not shown in FIG. 3A).

In operation, the diagnostic device of FIGS. 3A-3E is configured to utilize a pumping tool 314 to move fluid between the reagent reservoirs as described further below. The pumping tool in the example of FIGS. 3A-3E is designed to be operable by a user's hand (e.g., by a single finger) to slide the pumping tool 314 back and forth on one of the three ‘lanes’ and to thereby move liquid for each phase of the assay. The pumping tool can be moved between lanes to pump an appropriate part of the device for each phase as described below.

In some embodiments, first reagent reservoir 304 comprises a first set of reagents (e.g., one or more lysis reagents). In some embodiments, the first set of reagents comprises one or more reagents in solid form (e.g., lyophilized, dried, crystallized, air jetted). In some embodiments, the first set of reagents comprises one or more reagents in liquid form (e.g., in solution). In some embodiments, the contents of first reagent reservoir 304 are shielded from the environment by a removable cap and/or a breakable seal (e.g., a foil seal, a polymeric film).

In some instances, first reagent reservoir 304 is fluidically connected (e.g., directly fluidically connected) to a first fluidic channel. In some embodiments, the first fluidic channel is also fluidically connected (e.g., directly fluidically connected) to second reagent reservoir 306. In some embodiments, second reagent reservoir 306 comprises a second set of reagents (e.g., one or more nucleic acid amplification reagents). In some embodiments, the second set of reagents comprises one or more reagents in solid form (e.g., lyophilized, dried, crystallized, air jetted). In some embodiments, the second set of reagents comprises one or more reagents in liquid form (e.g., in solution). In some embodiments, the contents of second reagent reservoir 306 are shielded from the environment by a removable cap and/or a breakable seal (e.g., a foil seal, a polymeric film).

In some instances, second reagent reservoir is fluidically connected (e.g., directly fluidically connected) to a second fluidic channel. In some embodiments, the second fluidic channel is also fluidically connected to third reagent reservoir 308. In some embodiments, third reagent reservoir 308 comprises a third set of reagents (e.g., a dilution buffer).

In some instances, second reagent reservoir 306 is fluidically connected (e.g., directly fluidically connected) to vent path 310. In some embodiments, vent path 310 is configured to maintain a desired pressure in second reagent reservoir 306. As shown in FIGS. 3A-3E, vent path 310 may be substantially serpentine (e.g., comprises one or more turns, comprises two or more turns). In some cases, the serpentine nature of the vent path may discourage liquid contents of second reagent reservoir 306 from exiting cartridge 300. In the example of FIGS. 3A-3E, vent path 310 is coupled to a filter 331 that is permeable to gases but not liquids to allow air/gas to vent out of the system without allowing liquid to escape.

In the example of FIGS. 3A-3E, cartridge 300 comprises detection region 312. In some embodiments, detection region 312 is fluidically connected (e.g., directly fluidically connected) to a third fluidic channel. In some embodiments, second reservoir 306 is also fluidically connected (e.g., directly fluidically connected) to the third fluidic channel. In some embodiments, detection region 312 is fluidically connected (e.g., directly fluidically connected) to vent path 306. In some embodiments, detection region 312 comprises a lateral flow assay strip configured to detect one or more target nucleic acid sequences. In some embodiments, the lateral flow assay strip comprises one or more test lines comprising one or more capture reagents (e.g., immobilized antibodies) configured to detect one or more target nucleic acid sequences. In some embodiments, the lateral flow assay strip comprises one or more control lines. In some instances, detection region 312 comprises an angled pocket housing the lateral flow assay strip. In certain cases, the angled pocket may facilitate insertion of the lateral flow assay strip during manufacturing and/or may ensure that fluids from second reagent reservoir 306 are introduced to an input end (e.g., sample pad) of the lateral flow assay strip.

In the example of FIGS. 3A-3E, cartridge 300 comprises pumping tool 314. In some embodiments, pumping tool 314 comprises a peristaltic pump (e.g., a roller pump) and/or a reciprocating pump. In certain instances, for example, pumping tool 314 comprises a roller component. As shown in FIGS. 3A-3E, pumping tool 314 may be positioned above cartridge body 302. In some embodiments, cartridge 300 further comprises one or more pump lanes. A pump lane generally refers to at least a portion of a fluidic channel along which a user can direct pumping tool 314 to move. For example, as shown in FIGS. 3B, 3D, and 3E, cartridge 300 may comprise first pump lane 324 (left, corresponding to a portion of the first fluidic channel), second pump lane 326 (center, corresponding to a portion of the second fluidic channel), and third pump lane 328 (right, corresponding to a portion of the third fluidic channel). In some cases, pump lanes 324, 326, and 328 are formed by openings in seal plate 316, which may be attached to cartridge body 302 by one or more fasteners (e.g., one or more screws, nails, clamps, and/or bolts), one or more adhesives, one or more interlocking components, or any other type of attachment. In some embodiments, seal plate 316 and cartridge body 302 are configured to snap together without additional fasteners. In some embodiments, a membrane 318 (e.g., a peristaltic membrane) is positioned between seal plate 316 and cartridge body 302. In certain cases, at least one (and, in some cases, all) of pump lanes 324, 326, and 328 comprise at least one surface (e.g., one or more bottom and/or side surfaces) formed by cartridge body 302 (e.g., grooves within cartridge body 302) and at least one surface (e.g., a top surface) formed by membrane 318. Membrane 318 may be formed from any suitable material. A non-limiting example of a suitable membrane material is silicone. In some cases, at least one (and, in some cases, all) of pump lanes 324, 326, and 328 comprise one or more valves (e.g., passive valves) and/or bypass sections. In some cases, the one or more valves and/or bypass sections are configured to direct fluid flow in a particular direction (e.g., one-way fluid flow). In some embodiments, one or more pump lanes (e.g., pump lane 326) may optionally be blocked off.

In the example of FIGS. 3A-3E, cartridge 300 comprises an integrated heater 320. In some embodiments, heater 320 is a PCB heater. The PCB heater may comprise a bonded PCB with a microcontroller, thermistors, and resistive heaters. In some embodiments, the heater comprises a USB- and/or battery-powered heater. In some embodiments, one or more heating elements of heater 320 may be in thermal communication with first reagent reservoir 304 and/or second reagent reservoir 306. In certain instances, for example, one or more heating elements of heater 320 are located under first reagent reservoir 304 and/or second reagent reservoir 306. In some cases, heater 320 runs a first heating protocol (e.g., a lysis heating protocol) and/or a second heating protocol (e.g., a nucleic acid amplification protocol). In some instances, heater 320 is pre-programmed to run the first heating protocol and/or the second heating protocol.

In operation, a user may use a swab to collect a sample from a subject (e.g., the user, a friend or family member of the user, or any other human or animal subject). In certain instances, the user may insert the swab into a nasal cavity (e.g., anterior nares) or an oral cavity of the subject. In some cases, the user may then expose the contents of first reagent reservoir 304. In some instances, the user may remove a removable cap covering first reagent reservoir 304. In some instances, the user may use a puncturing tool (e.g., a sterile puncturing tool) to puncture a film covering first reagent reservoir 304. The user may then insert a portion of the swab bearing a collected sample into first reagent reservoir 304. The swab may be stirred in the fluidic contents of first reagent reservoir 304 to promote transfer of at least a portion of the sample to the fluidic contents of first reagent reservoir 304. In some embodiments, chemical lysis may be performed by one or more lysis reagents (e.g., enzymes, detergents) in first reagent reservoir 304. In some embodiments, thermal lysis may be performed by heater 320. In certain cases, for example, heater 320 may heat first reagent reservoir 304 according to a first heating protocol (e.g., a lysis heating protocol). In this manner, one or more cells within the sample may be lysed.

In some embodiments, the user may push pumping tool 314 along first pump lane 324. In some cases, pushing pumping tool 314 along first pump lane 324 may transport at least a portion of the fluidic contents of first reagent reservoir 304 (e.g., comprising a lysate) to second reagent reservoir 306. In some instances, second reagent reservoir 306 comprises a second set of reagents (e.g., one or more nucleic acid amplification reagents). The second set of reagents may comprise one or more reagents in solid form and/or one or more reagents in liquid form. In some embodiments, second reagent reservoir 306 comprises one or more nucleic acid amplification reagents in solid form (e.g., lyophilized, dried, crystallized, air jetted). In some instances, introduction of the fluidic contents of first reagent reservoir 304 to second reagent reservoir 306 may cause the one or more nucleic acid amplification reagents in solid form to dissolve. In certain cases, heater 320 may heat second reagent reservoir 306 according to a second heating protocol (e.g., a nucleic acid amplification heating protocol). In this manner, one or more target nucleic acid sequences may be amplified (if present within the sample). The amplified nucleic acids may be referred to as amplicons. In some cases, vent path 310 may allow a desired pressure to be maintained in second reagent reservoir 306 while preventing amplicon egress.

Optionally, in some cases, if additional fluid is needed in second reagent reservoir 306, fluidic contents (e.g., a dilution buffer) from third reagent reservoir 308 may be transported to second reagent reservoir 306 by moving pumping tool 314 along second pump lane 326. In some embodiments, if additional fluid is not needed, third reagent reservoir 308 (and second pump lane 326) may be removed from cartridge 300 and/or second pump lane 326 may be blocked off.

In some embodiments, the fluidic contents of second reagent reservoir 306 (e.g., amplicon-containing fluid) may be transported from second reagent reservoir 306 to detection region 312 by pushing pumping tool 314 along third pump lane 328. In this manner, at least a portion of the fluidic contents of second reagent reservoir 306 may be introduced into a first portion (e.g., sample pad) of a lateral flow assay strip in detection region 312. In some cases, the fluidic contents may flow through the lateral flow assay strip (e.g., via capillary action). In some cases, at least a portion of the lateral flow assay strip may be visible to the user, and the user may be able to determine whether or not one or more target nucleic acid sequences are present based on the formation (or lack thereof) of one or more opaque lines (or other markings) on the lateral flow assay strip.

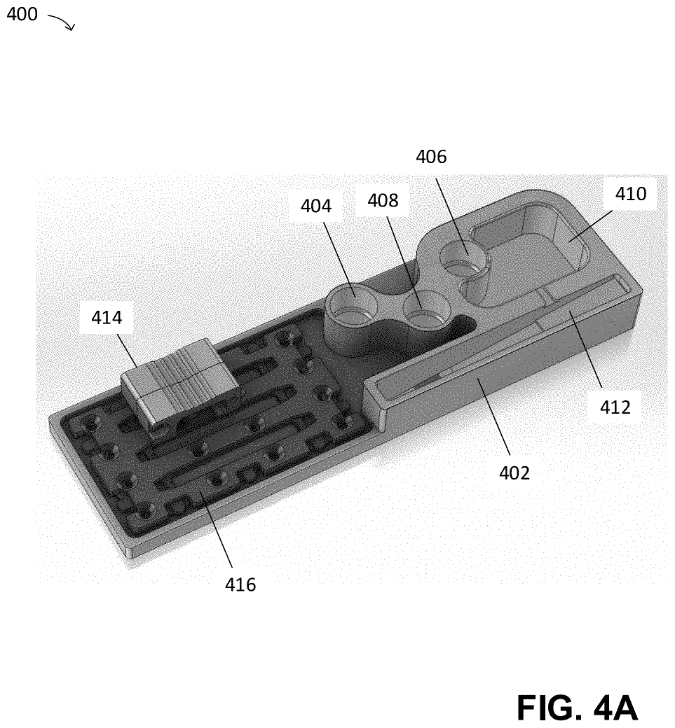

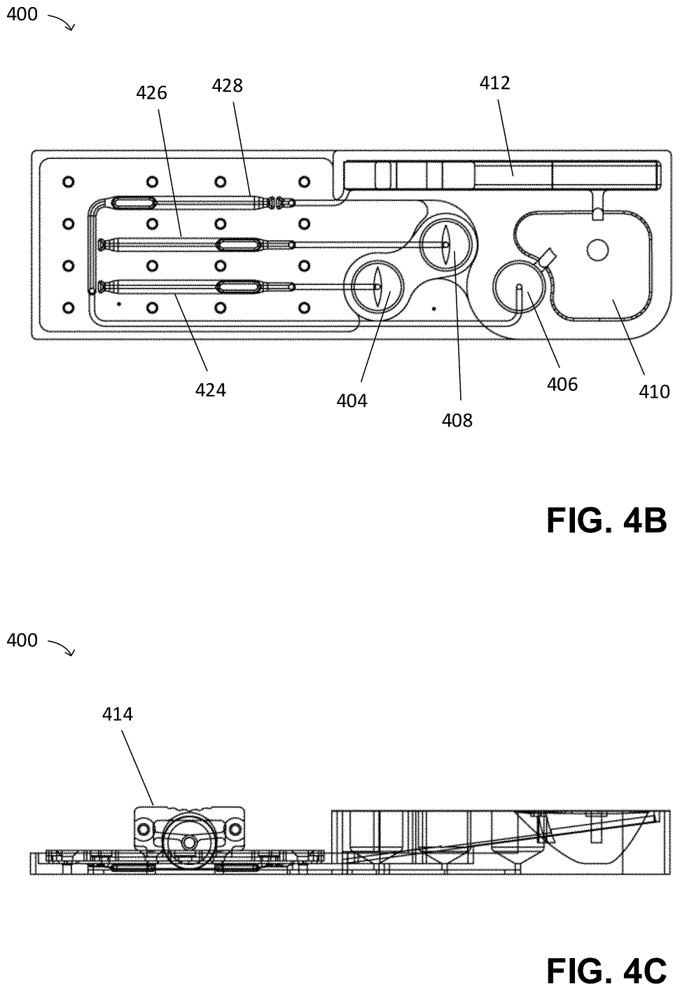

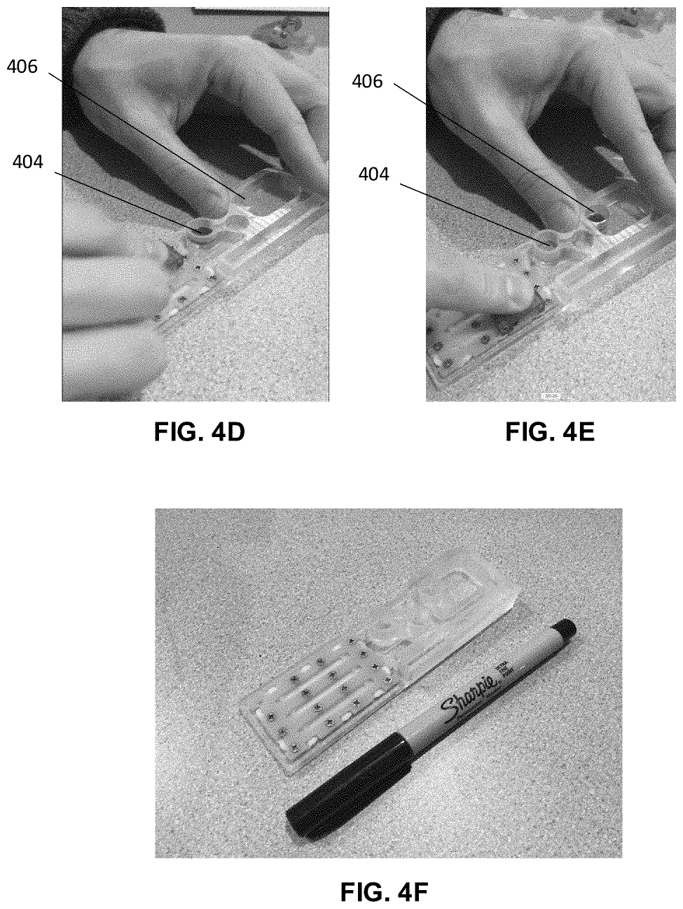

Many variations of the cartridge are possible. In some embodiments, a cartridge comprises a gas expansion reservoir instead of a vent path (e.g., a serpentine vent path). For example, FIGS. 4A-4F show cartridge 400 comprising a gas expansion reservoir 410. In addition to gas expansion reservoir 410, cartridge 400 further comprises first reagent reservoir 404, second reagent reservoir 406, third reagent reservoir 408, and detection region 412, all of which are formed within cartridge body 402. Cartridge 400 also comprises pumping tool 414, seal plate 416, and pumping lanes 424, 426, and 428. In some cases, expansion reservoir 410 is fluidically connected (e.g., directly fluidically connected) to second reagent reservoir 406. Like a vent path, gas expansion reservoir 410 may facilitate the maintenance of a desired pressure in second reagent reservoir 406.

FIGS. 4B-4F provide additional views of cartridge 400. FIG. 4B shows a back view of cartridge 400, and FIG. 4C shows a side view of cartridge 400. FIGS. 4D-4F show photographs illustrating fluid flow in cartridge 400. In FIG. 4D, a fluid is present in first reagent reservoir 404. By moving pumping tool 414, in FIG. 4E, the fluid has been transported from first reagent reservoir 404 to second reagent reservoir 406. FIG. 4F shows cartridge 400 with a marker for comparison.

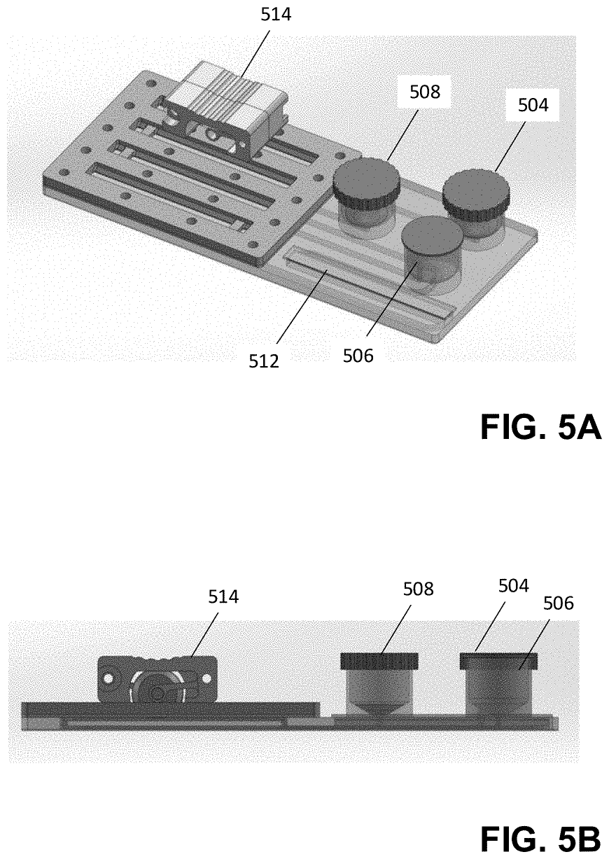

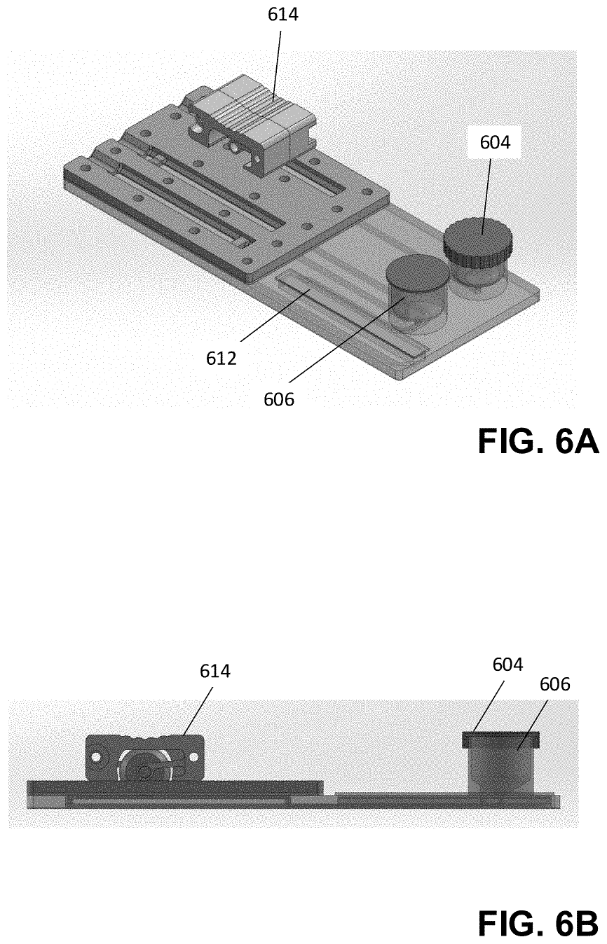

In some embodiments, a cartridge comprises a plurality of reservoirs with removable caps. One such example of a cartridge that comprises three reagent reservoirs is shown in FIGS. 5A-5B, which depict a cartridge 500 comprising first reagent reservoir 504, second reagent reservoir 506, third reagent reservoir 508, detection region 512, and pumping tool 514. As shown in FIG. 5A, first reagent reservoir 504, second reagent reservoir 506, and third reagent reservoir 508 all have removable caps. In some embodiments, a cartridge comprises two reagent reservoirs, and one or both of the reagent reservoirs comprise removable caps. For example, FIGS. 6A-6B show cartridge 600 comprising first reagent reservoir 604 and second reagent reservoir 606, as well as pumping tool 614. As shown in FIGS. 6A-6B, first reagent reservoir 604 and second reagent reservoir 606 both comprise removable caps.

In some embodiments, a cartridge comprises a single reagent reservoir. In some cases, the single reagent reservoir comprises one or more reagents. In some embodiments, for example, the single reagent reservoir comprises one or more lysis reagents and/or one or more nucleic acid amplification reagents. In some embodiments, lysis is performed via thermal lysis, and the single reagent reservoir does not comprise lysis reagents. The one or more reagents may be in solid form (e.g., lyophilized, dried, crystallized, air jetted) or liquid form.

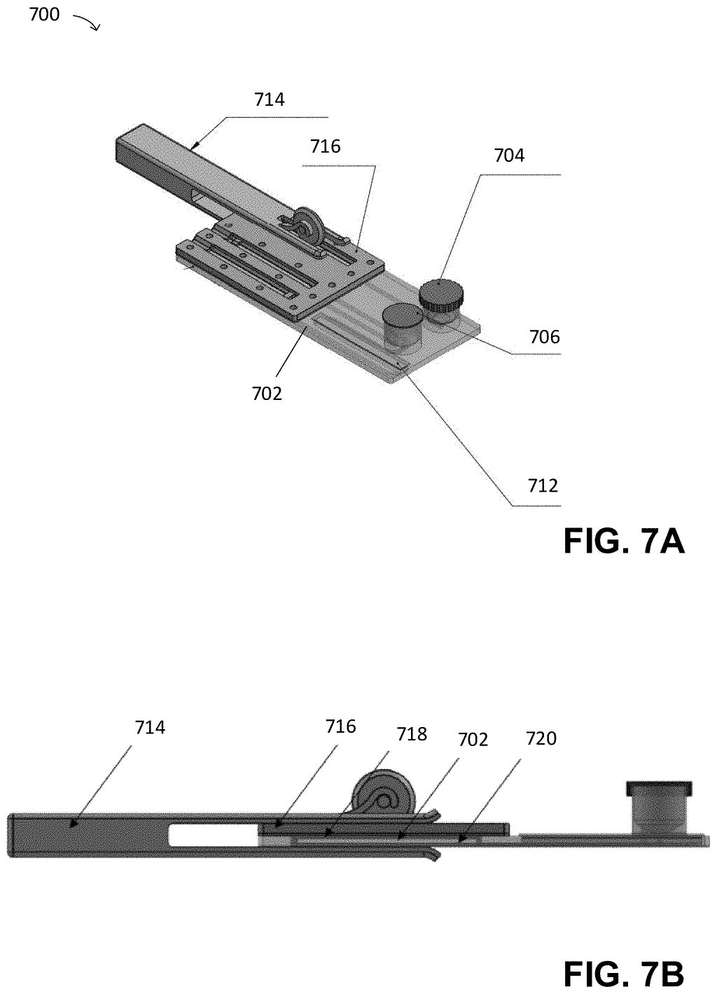

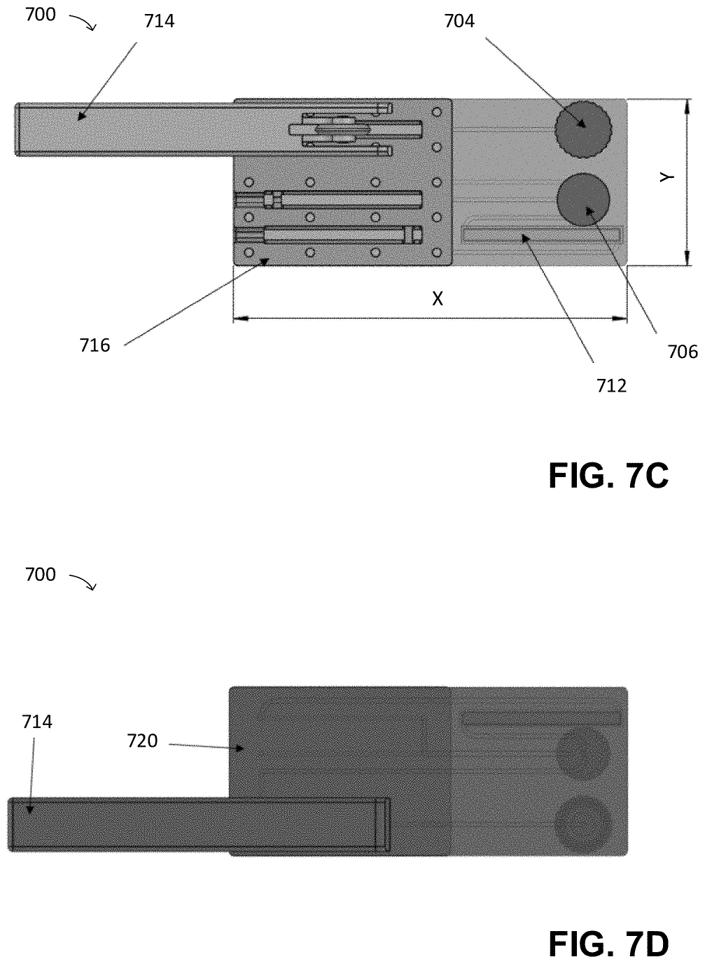

In some embodiments, a cartridge comprises a pumping tool that wraps around the body of the cartridge instead of sitting above the body of the cartridge. FIGS. 7A-7D show an illustrative embodiment comprising cartridge 700, which comprises wraparound pumping tool 714. Wraparound pumping tool 714 comprises a top component, a bottom component, and a rolling component. In certain cases, wraparound pumping tool 714 is designed to be attached to the cartridge body 702 without any fasteners.

In addition to wraparound pumping tool 714, cartridge 700 comprises cartridge body 702 comprising first reagent reservoir 704, second reagent reservoir 706, and detection region 712. In addition, cartridge 700 comprises seal plate 716. FIG. 7B shows a cross-sectional view of cartridge 700. From FIG. 7B, it can be seen that at least a portion of cartridge 700 (e.g., the portion comprising one or more pump lanes) comprises a top layer comprising seal plate 716, a second layer comprising membrane 718 (e.g., a peristaltic membrane), a third layer comprising cartridge body 702, and a bottom layer 720. FIG. 7C shows a top view of cartridge 700, and FIG. 7D shows a bottom view of cartridge 700.

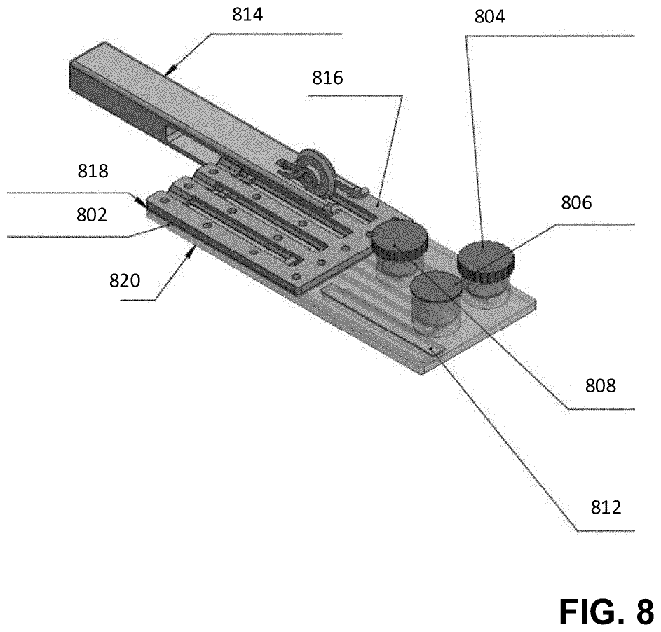

FIG. 8 also shows a cartridge comprising a wraparound pumping tool. In FIG. 8, cartridge 800 comprises cartridge body 802 comprising first reagent reservoir 804, second reagent reservoir 806, third reagent reservoir 808, and detection region 812. As shown in FIG. 8, first reagent reservoir 804, second reagent reservoir 806, and third reagent reservoir 808 may comprise removable caps. In addition, FIG. 8 comprises seal plate 816, membrane 818, and bottom layer 820.

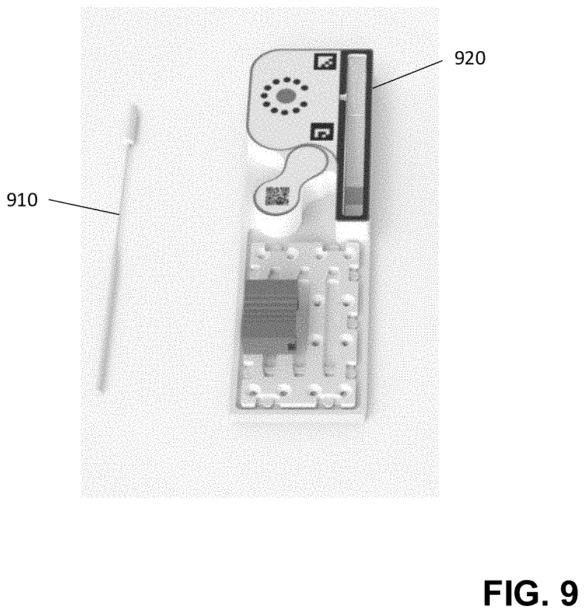

In some cases, a cartridge may be a component of a diagnostic system. For example, FIG. 9 illustrates an exemplary diagnostic system 900 comprising sample-collecting swab 910 and cartridge 920. In some embodiments, the diagnostic system may be used with an electronic device (e.g., a smartphone, a tablet) and associated software (e.g., a mobile application). In some embodiments, for example, the software may provide instructions for using the cartridge, may read and/or analyze results, and/or report results. In certain instances, the electronic device may communicate with the cartridge (e.g., via a wireless connection).

Having thus described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the spirit and scope of the invention. Further, though advantages of the present invention are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

By way of example, fluid reservoirs in any of the above-described devices or systems may be configured to hold relatively small volumes, such as a volume of at least 10 μL, at least 20 μL, at least 50 μL, at least 100 μL, at least 200 μL, at least 500 μL, at least 1 mL, at least 2 mL, at least 5 mL, at least 10 mL, at least 20 mL, or at least 50 mL. Reservoirs (e.g., reagent reservoir, gas expansion reservoir) may also have any suitable shape, including a cylindrical shape, a cubic shape, a cuboidal shape, a prismatic shape, or a conical shape. Each reservoir (e.g., reagent reservoir, gas expansion reservoir) may also have any suitable cross-sectional shape. For example, in some cases, one or more reservoirs may have a cross-section that is rectangular, square, triangular, circular, U-shaped, serpentine, hexagonal, or irregularly shaped.

As another example, fluidic channels in any of the above-described devices or systems may be configured with a cross-sectional dimension (e.g., a diameter, a width) that falls within one of the ranges listed below, as measured perpendicular to the direction of fluid flow. In some embodiments, one or more fluidic channels have a maximum cross-sectional dimension of about 5 mm or less, about 2 mm or less, about 1 mm or less, about 800 μm or less, about 500 μm or less, about 200 μm or less, about 100 μm or less, about 80 μm or less, about 50 μm or less, about 20 μm or less, or about 10 μm or less. Fluidic channels may also have a channel width-to-depth ratio of at least about 0.1, at least about 0.2, at least about 0.5, at least about 1, at least about 2, at least about 5, or at least about 10. Fluidic channels may also have a length of at least 1 cm, at least 2 cm, at least 5 cm, at least 10 cm, or at least 20 cm.

Moreover, the diagnostic devices, systems, and methods described herein may be used to detect the presence or absence of any target nucleic acid sequence (e.g., from any pathogen of interest). Target nucleic acid sequences may be associated with a variety of diseases or disorders, as described below. In some embodiments, the diagnostic devices, systems, and methods are used to diagnose at least one disease or disorder caused by a pathogen. In certain instances, the diagnostic devices, systems, and methods are configured to detect a nucleic acid encoding a protein (e.g., a nucleocapsid protein) of SARS-CoV-2, which is the virus that causes COVID-19. In some embodiments, the diagnostic devices, systems, and methods are configured to identify particular strains of a pathogen (e.g., a virus). In some embodiments, a diagnostic device comprises a lateral flow assay strip comprising a first test line configured to detect a nucleic acid sequence of SARS-CoV-2 and a second test line configured to detect a nucleic acid sequence of a SARS-CoV-2 virus having a D614G mutation (i.e., a mutation of the 614^{th} amino acid from aspartic acid (D) to glycine (G)) in its spike protein. In some embodiments, one or more target nucleic acid sequences are associated with a single-nucleotide polymorphism (SNP). In certain cases, diagnostic devices, systems, and methods described herein may be used for rapid genotyping to detect the presence or absence of a SNP, which may affect medical treatment.

In some embodiments, the diagnostic devices, systems, and methods are configured to detect a target nucleic acid sequence of a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In some embodiments, the viral pathogen is SARS-CoV-2 and/or SARS-CoV-2 D614G. In some embodiments, the viral pathogen is an influenza virus. The influenza virus may be an influenza A virus (e.g., H1N1, H3N2) or an influenza B virus.

Other viral pathogens include, but are not limited to, adenovirus; Herpes simplex, type 1; Herpes simplex, type 2; encephalitis virus; papillomavirus (e.g., human papillomavirus); Varicella zoster virus; Epstein-Barr virus; human cytomegalovirus; human herpesvirus, type 8; BK virus; JC virus; smallpox; polio virus; hepatitis A virus; hepatitis B virus; hepatitis C virus; hepatitis D virus; hepatitis E virus; human immunodeficiency virus (HIV); human bocavirus; parvovirus B19; human astrovirus; Norwalk virus; coxsackievirus; rhinovirus; Severe acute respiratory syndrome (SARS) virus; yellow fever virus; dengue virus; West Nile virus; Guanarito virus; Junin virus; Lassa virus; Machupo virus; Sabiá virus; Crimean-Congo hemorrhagic fever virus; Ebola virus; Marburg virus; measles virus; mumps virus; rubella virus; Hendra virus; Nipah virus; Rabies virus; rotavirus; orbivirus; Coltivirus; Hantavirus; Middle East Respiratory Coronavirus; Zika virus; norovirus; Chikungunya virus; and Banna virus.

The above-described embodiments of the technology described herein can be implemented in any of numerous ways. Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

The term “substantially” may be used to refer to values that are within ±20% of a comparative measure in some embodiments, within ±10% in some embodiments, within ±5% in some embodiments, and yet within ±2% in some embodiments. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. An integrated single-use device for performing a nucleic acid diagnostic test, the device comprising:
a lysis chamber for accepting a sample suspected of comprising a target nucleic acid sequence, wherein the lysis chamber comprises a lysis buffer;
an amplification chamber operably connected to the lysis chamber through at least a first channel;
a readout strip operably connected to the amplification chamber; and
a pumping tool configured to be operated to transport at least some of the sample from the lysis chamber through the first channel and into the amplification chamber of the device.

2. The device of claim 1, wherein the readout strip is a lateral flow strip comprising one or more reagents for CRISPR/Cas detection.

3. The device of claim 2, wherein the one or more reagents for CRISPR/Cas detection comprise one or more guide nucleic acids.

4. The device of claim 3, wherein the one or more guide nucleic acids include at least one guide nucleic acid configured to recognize a specific target sequence of a pathogen, and wherein the pathogen is SARS-CoV-2.

5. The device of claim 2, wherein the one or more reagents for CRISPR/Cas detection comprise one or more programmable nucleases.

6. The device of claim 5, wherein the one or more programmable nucleases includes at least one Cas protein.

7. The device of claim 2, wherein the one or more reagents for CRISPR/Cas detection comprise a plurality of guide nucleic acids and a plurality of programmable nucleases.

8. The device of claim 2, wherein the lateral flow strip comprises a plurality of test lines that each comprise a reagent configured to detect a respective target nucleic acid.

9. The device of claim 8, wherein each of the plurality of test lines is configured to change color when the respective target nucleic acid is detected.

10. The device of claim 2, wherein the lateral flow strip comprises at least one control line comprising a reagent configured to detect a nucleic acid expected to be present in all samples produced by humans.

11. The device of claim 1, wherein the device is configured to transport the at least some of the sample to the readout strip from the amplification chamber via capillary action.

12. The device of claim 1, wherein the amplification chamber comprises lyophilized amplification reagents.

13. The device of claim 1, further comprising a heat source, wherein the heat source is configured to heat the lysis chamber to at least 65° C.

14. The device of claim 1, wherein the pumping tool comprises a seal plate, an engaged roller, and at least two pump lanes.

15. The device of claim 14, wherein the engaged roller is configured to be controlled by a user.

16. The device of claim 14, wherein the engaged roller moves along each pump lane to move the sample from the lysis chamber to the amplification chamber and the amplification chamber to the readout strip.

17. The device of claim 1, wherein the readout strip comprises a test control line, a human sample control line, and a first disease test line, and wherein the first disease comprises a coronavirus.

18. A system comprising the device of claim 1 and at least one computer readable medium comprising instructions that, when executed, cause a computing device to image the device and present results of the diagnostic test based on the image.

19. An integrated single-use device for performing a nucleic acid diagnostic test, the device comprising:
a cartridge comprising:
a lysis chamber for accepting a sample suspected of comprising a target nucleic acid sequence, wherein the lysis chamber comprises a lysis buffer; and
an amplification chamber operably connected to the lysis chamber through at least a first channel of the cartridge, wherein the amplification chamber comprises lyophilized amplification reagents,
wherein the first channel and the amplification chamber are enclosed within the cartridge; and
a pumping tool configured to be operated to transport at least some of the sample from the lysis chamber through the first channel and into the amplification chamber of the device.

20. The device of claim 19, further comprising a heat source, wherein the heat source is configured to heat the lysis chamber to at least 65° C.
